(19) United States
(12) Patent Application Publication
(10) Pub. No.: US 2026/0315570 A1
(43) Pub. Date: Oct. 15, 2026

(54) METHOD AND COMPOSITION FOR ASSESSING AND ENHANCING THE DURABILITY OF ANTIBODY RESPONSE TO VACCINATION

(71) Applicant: The Board of Trustees of the Leland Stanford Junior University, Stanford, CA (US)

(72) Inventors: Bali Pulendran, Redwood City, CA (US); Sheng-Yang Wu, Redwood City, CA (US)

(21) Appl. No.: 19/481,115

(22) PCT Filed: May 3, 2024
(86) PCT No.: PCT/US2024/027748

§ 371 Date: Nov. 3, 2025

Related U.S. Application Data
(60) Provisional application No. 63/464,389, filed on May 5, 2023.

(51) Int. Cl. A61K 38/19; A61K 31/4152; A61K 31/4155; A61K 31/426; A61K 39/215; A61K 39/39; A61P 31/14; A61P 37/04

(52) U.S. Cl. CPC A61K 38/196; A61K 31/4152; A61K 31/4155; A61K 31/426; A61K 39/215; A61K 39/39; A61P 31/14; A61P 37/04; C12N 2770/20034

(57) ABSTRACT

Methods are provided herein for enhancing the durability of response to vaccines.

### CROSS REFERENCE TO OTHER APPLICATIONS

This application claims the benefit of U.S. Provisional Application No. 63/464,389 filed May 5, 2023, the contents of which are hereby incorporated by reference in its entirety.

### BACKGROUND

The limited duration of humoral responses to vaccination is a key issue in the fight against infectious diseases, as antibody levels wane over time, leaving individuals vulnerable to reinfection. Individuals who display persistent humoral response to vaccination have been shown to have upregulated transcriptional signatures in platelet activation.

Thrombopoietic activity refers to the physiological process by which the body produces platelets. During thrombopoiesis, immature megakaryocytes increase their ploidy and differentiate into mature polyploid megakaryocytes, which then undergo cytoplasmic fragmentation to give rise to small anucleate cell fragments called platelets. Newly generated platelets preserve RNA content from megakaryocytes, thus are named reticulated platelets. As platelets mature, cytosolic RNA gradually degrades, resulting in mature RNA-free platelets. (Mauro Buttarello and Mario Plebani. Am J Clin Pathol, 2008.) Thrombopoietin (TPO) is the primary cytokine that regulates thrombopoietic activity. TPO binds to its receptor, c-Mpl, on the surface of megakaryocytes, leading to activation of signaling pathways that promote the proliferation, differentiation, and cytoplasmic fragmentation of megakaryocytes (Françoise Wendling et al. Nature, 1994).

### SUMMARY

Compositions and methods are provided for enhancing the durability of an antibody response to an immunogen, e.g. a vaccine. Durability is enhanced through the administration of a thrombopoietin (TPO) agent, e.g. a TPO agonist, in combination with an immunogen. A TPO agonist may include without limitation: thrombopoietin protein (TPO), e.g. human TPO; mRNA- or DNA-based vectors expressing thrombopoietin; small or large molecules that activate thrombopoietic activity via cMPL, including thrombopoietin receptor agonists (TPO-RA) such as eltrombopag, avatrombopag, lusutrombopag, hetrombopag, romiplostim; and the like. By enhancing both the magnitude and durability of antibody responses to an immunogen, improved methods are provided for developing long-term protection against infectious diseases or tumor antigens, addressing a critical need in the field.

The results presented herein demonstrate that durability of an antibody response is associated with the status of megakaryocytes; and thrombopoietic activity in the bone marrow. Thrombopoietic activity may contribute to long lived plasma cell (LLPC) survival. Administration of a TPO agonist, in combination with an immunogen, enhances thrombopoietic activity at the time of immunization, and significantly increases antibody levels specific for the immunogen for an extended prior of time. Enhancing thrombopoietic activity augments the magnitude and prolongs the duration of antibody responses to an immunogen.

In some embodiments, a method is provided for immunization, comprising administering an effective dose of a TPO agonist prior to the administration of an immunogen, concurrently with an immunogen, or following administration of an immunogen. In some embodiments the TPO agonist is administered from about 1, about 2, about 3, about 5, to about 10 days prior, and/or about about 1, about 2, about 3, about 5, to about 10 days following administration of the immunogen, and may be administered on the same day as the immunogen. An effective dose may be, for example, equivalent to from about 1 to 1000 μg/kg TPO protein.

The TPO agonist may be administered as a single dose or multiple doses, as appropriate for the specific agent, e.g. 1, 2, 3, 4, 5, or more doses. The TPO agonist may be administered in combination with an initial, “priming” dose of immunogen; may be administered in combination with one or more additional, “booster” doses of immunogen; or may be administered with both.

The methods of the disclosure can increase the durability of an response to an immunogen. Durability may be calculated, for example, as a “residual”, which is calculated as the difference between observed antibody titers at day 180 and the antibody titers predicted at day 180 based on the simple linear correlation between the day 42 titers (peak) and day 180 titers. The residual may be a positive number with the inclusion of a thrombopoietin agent, e.g. at least 0.5, at least 1, at least 2, up to 4 or 4.

In some embodiments a vaccine composition is provided, comprising an effective dose of an immunogen; and an effective dose of a TPO agonist. The agents may be co-formulated, or may be separately formulated. One or both of the vaccine components may be provided in a pharmaceutically acceptable adjuvant. One or both of the vaccine components may be provided in lyophilized form. An adjuvant may be optionally provided. The TPO agonist may include without limitation: thrombopoietin protein; mRNA- or DNA-based vectors expressing thrombopoietin; small or large molecules that activate thrombopoietic activity via cMPL including thrombopoietin receptor agonists (TPO-RA) such as eltrombopag, avatrombopag, lusutrombopag, hetrombopag, romiplostim; and the like.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIGS. 1A-1J. (A) Experimental timeline of platelet staining and HAI assay in seasonal influenza vaccination. Subjects were vaccinated with one dose of TIV (Fluzone®, Sanofi Pasteur Inc., in 2010-11 season). (B) The gating strategy for cell-free platelets in thawed PBMCs of human subjects in 2010-2011 seasonal influenza vaccination. (C) Scatterplots of the day 180/day 28 HAI residual versus the day 7/day 0 log₂ fold change of RNA content (median RNA dye intensity) in whole platelet and % RNA⁺ platelets. (D) Experimental timeline of platelet staining and neutralizing assay in Rhesus macaques immunized with RBD-NP or hexa-Pro-NP admixed with_AS03. (E) Line graph of the kinetics of pseudoviral_neutralizing antibody titers in serum. (F) Scatterplots of the day180/day42 nAb residual versus the day28 (d7)/Baseline (d0) log₂ FC of RNA content in whole platelet and % RNA⁺ platelets. (G) Experimental timeline of platelet staining, ELISA, and ELISPOT in C57BL/6J mice immunized with SARS-COV-2 (2019-nCOV) Spike admixed with AS03. (H) Line graph of the kinetics of anti-Spike binding antibody titers in serum. (I, J) Scatterplots of the day 7/day 0 log 2 FC of RNA content in whole platelets and % RNA⁺ platelets versus the day 42/day 7 residual Ab (I) and the bone marrow Spike+IgG ASC numbers (J).

FIGS. 2A-2C. A. Experimental timeline of TPO injection and Spike AS03 immunization in WT and cMpIKO mice. (B, C) Line graph of anti-Spike binding antibody titers and fold change of antibody response in serum in two independent experiments.

### DETAILED DESCRIPTION

Compositions and methods are provided for enhancing the durability of an antibody response to an immunogen.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “adjuvant” generally refers to a composition that increases the humoral or cellular immune response of an individual. Adjuvants of interest stimulate the immune system, and increase responsiveness or durability of response to a co-administered antigen. Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249:1527, 1990 and Hanes, Advanced Drug Delivery Reviews 28:97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

As vaccine components that enhance the magnitude, breadth, and durability of the immune response, adjuvants are powerful tools for modern vaccine development. By enabling antigen-sparing, adjuvants also allow for more rapid vaccine production, a critical factor during response to pandemics. For more than 70 years, insoluble aluminum salts (alum) were the only licensed adjuvant, however in the past 3 decades there has been a large expansion in adjuvants available in licensed vaccines. These include oil-in-water emulsion-based adjuvants (MF59, AS03), adjuvants containing the TLR4 agonist 3-O-desacyl-4′-monophosphoryl lipid A (MPL) (AS01, AS04), and CpG 1018, a TLR9 agonist CpG oligonucleotide. Many of these adjuvants, including AS03, MF59, and CpG 1018, have been made available by their owners for use in COVID-19 vaccines, and at least 10 developers have indicated plans to create adjuvanted COVID-19 vaccines. Despite this large growth in adjuvant technology, in many cases the molecular mechanisms by which these adjuvants boost immune responses to vaccination remain unclear.

AS03 is a squalene-based oil-in-water emulsion containing α-tocopherol (Vitamin E), and has been shown to increase the breadth and magnitude of CD4+ T cell and antibody responses against multiple strains of influenza, even compared with MF59. Recent work in mice demonstrated that AS03 induced alterations in expression of lipid metabolism-related genes in the draining lymph nodes, as well as increased endoplasmic reticulum (ER) stress in macrophages, which drove elevated cytokine production and improved antibody responses. Additionally, the similar oil-in-water emulsion-based adjuvant MF59 has been shown to induce a local release of extracellular ATP, and to depend functionally on MyD88 in an inflammasome-independent fashion, suggesting that these type of adjuvants produce some degree of cell injury or stress that result in release of damage-associated molecular patterns (DAMPs) and induction of innate immune responses. However, the molecular pathways through which AS03 promotes these responses remain poorly defined.

In addition to boosting the initial immune response to vaccination, adjuvants, including TPO agents, can also improve the longevity of the resulting immunity. Whereas some vaccines, particularly live viral vaccines such as smallpox or yellow fever, can induce lifelong antibody responses, others, such as those against pertussis and influenza, only promote transient responses and immunity that wanes over time, resulting in a loss of protection and need for booster vaccinations. With regards to humoral immunity, long-lived plasma cells have been identified as key mediators of durable antibody responses, but the mechanisms required to drive robust long-lived plasma cell differentiation and persistent antibody responses to vaccination are not well understood.

“Antigen” or “immunogen” refers to any substance that stimulates an immune response. The term includes killed, inactivated, attenuated, or modified live bacteria, viruses, or parasites, and may include tumor-associated antigens. The term antigen also includes polynucleotides, polypeptides, recombinant proteins, synthetic peptides, protein extract, cells (including bacterial cells), tissues, polysaccharides, or lipids, or fragments thereof, individually or in any combination thereof. The term antigen also includes antibodies, such as anti-idiotype antibodies or fragments thereof, and to synthetic peptide mimotopes that can mimic an antigen or antigenic determinant (epitope).

The terms “subject,” “individual,” and “patient” are used interchangeably herein to refer to a mammal being assessed for response. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc. The methods of the invention can be applied for veterinary purposes.

The terms “therapeutic agent”, “therapeutic capable agent” or “treatment agent” are used interchangeably and refer to a molecule or compound that confers some beneficial effect upon administration to a subject, including vaccines and vaccine adjuvants. The beneficial effect includes induction of a therapeutic immune response, enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose will vary depending on the particular agent chosen, the dosing regimen to be followed, whether it is administered in combination with other compounds, timing of administration, the tissue to be imaged, and the physical delivery system in which it is carried.

“Suitable conditions” shall have a meaning dependent on the context in which this term is used. That is, when used in connection with an antibody, the term shall mean conditions that permit an antibody to bind to its corresponding antigen. When used in connection with contacting an agent to a cell, this term shall mean conditions that permit an agent capable of doing so to enter a cell and perform its intended function. In one embodiment, the term “suitable conditions” as used herein means physiological conditions.

“In combination with”, “combination therapy” and “combination products” refer, in certain embodiments, to the concurrent administration to a patient of a first therapeutic (i.e., first therapeutic agent) and the compounds as used herein. When administered in combination, each component can be administered at the same time or sequentially in any order at different points in time. Thus, each component can be administered separately but sufficiently closely in time so as to provide the desired therapeutic effect. First therapeutic agents contemplated for use with the methods of the present invention include any other agent for use in immunization.

“Concomitant administration” of a known therapeutic agent with a pharmaceutical composition of the present invention means administration of the therapeutic agent and inhibitor agent at such time that both the known therapeutic agent and the composition of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention. Therapeutic agents contemplated for concomitant administration according to the methods of the present invention include any other agent for use in immunization.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient ”means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).) The term “inflammatory” response is the development of a humoral (antibody mediated) and/or a cellular response, which cellular response may be mediated by antigen-specific T cells or their secretion products), and innate immune cells. An “immunogen” is capable of inducing an immunological response against itself on administration to a mammal or due to autoimmune disease.

The term “vaccine”, as used herein, is defined in accordance with the pertinent art and relates to a composition that induces or enhances the protective immunity of an individual to a particular disease caused by a pathogen. Without wishing to be bound by theory, it is believed that a protective immunity arises from the generation of neutralizing antibodies, or from the activation of cytotoxic cells of the immune system, or both. In order to induce or enhance a protective immunity, a vaccine comprises as an immunogenic antigen a part of the pathogen causing said disease or a nucleic acid molecule encoding this immunogenic antigen. Upon contact with the immunogenic antigen, the immune system of the individual is triggered to recognise the immunogenic antigen as foreign and to destroy it. The immune system subsequently remembers the contact with this immunogenic antigen, so that at a later contact with the disease-causing pathogen an easy and efficient recognition and destruction of the pathogen is ensured.

Some examples of bacteria causing disease for which immune responsiveness may be obtained include, for example, Aceinetobacter calcoaceticus, Acetobacter paseruianus, Actinobacillus pleuropneumoniae, Aeromonas hydrophila, Alicyclobacillus acidocaldarius, Arhaeglobus fulgidus, Bacillus pumilus, Bacillus stearothermophillus, Bacillus subtilis, Bacillus thermocatenulatus, Bordetella bronchiseptica, Burkholderia cepacia, Burkholderia glumae, Campylobacter coli, Campylobacter fetus, Campylobacter jejuni, Campylobacter hyointestinalis, Chlamydia psittaci, Chlamydia trachomatis, Chlamydophila spp., Chromobacterium viscosum, Erysipelothrix rhusiopathieae, Listeria monocytogenes, Ehrlichia canis, Escherichia coli, Haemophilus influenzae, Haemophilus somnus, Helicobacter suis, Lawsonia intracellularis, Legionella pneumophilia, Moraxellsa sp., Mycobactrium bovis, Mycoplasma hyopneumoniae, Mycoplasma mycoides subsp. mycoides LC, Clostridium perfringens, Odoribacter denticanis, Pasteurella(Mannheimia ) haemolytica, Pasteurella multocida, Photorhabdus luminescens, Porphyromonas gulae, Porphyromonas gingivalis, Porphyromonas salivosa, Propionibacterium acnes, Proteus vulgaris, Pseudomnas wisconsinensis, Pseudomonas aeruginosa, Pseudomonas fluorescens C9, Pseudomonas fluorescensSIKW1, Pseudomonas fragi, Pseudomonas luteola, Pseudomonas oleovorans, Pseudomonas sp B11-1, Alcaliges eutrophus, Psychrobacter immobilis, Rickettsia prowazekii, Rickettsia rickettsia, Salmonella typhimurium, Salmonella bongori, Salmonella enterica, Salmonella dublin, Salmonella typhimurium, Salmonella choleraseuis, Salmonella newport, Serratia marcescens, Spirlina platensis, Staphlyoccocus aureus, Staphylococcus epidermidis, Staphylococcus hyicus, Streptomyces albus, Streptomyces cinnamoneus, Streptococcus suis, Streptomyces exfoliates, Streptomyces scabies, Sulfolobus acidocaldarius, Syechocystis sp., Vibrio cholerae, Borrelia burgdorferi, Treponema denticola, Treponema minutum, Treponema phagedenis, Treponema refringens, Treponema vincentii, Treponema palladium, and Leptospira species, such as the known pathogens Leptospira canicola, Leptospira grippotyposa, Leptospira hardjo, Leptospira borgpetersenii hardjo-bovis, Leptospira borgpetersenii hardjo-prajitno, Leptospira interrogans, Leptospira icterohaemorrhagiae, Leptospira pomona, and Leptospira bratislava, and combinations thereof.

Examples of viruses causing disease for which immune responsiveness may be obtained include, for example, SARS-Cov1, SARS-Cov2, and other coronaviruses, Avian herpesviruses, Bovine herpesviruses, Canine herpesviruses, Equine herpesviruses, Feline viral rhinotracheitis virus, Marek's disease virus, Ovine herpesviruses, Porcine herpesviruses, Pseudorabies virus, Avian paramyxoviruses, Bovine respiratory syncytial virus, Canine distemper virus, Canine parainfluenza virus, canine adenovirus, canine parvovirus, Bovine Parainfluenza virus 3, Ovine parainfluenza 3, Rinderpest virus, Border disease virus, Bovine viral diarrhea virus (BVDV), BVDV Type I, BVDV Type II, Classical swine fever virus, Avian Leukosis virus, Bovine immunodeficiency virus, Bovine leukemia virus, Bovine tuberculosis, Equine infectious anemia virus, Feline immunodeficiency virus, Feline leukemia virus (FeLV), Newcastle Disease virus, Ovine progressive pneumonia virus, Ovine pulmonary adenocarcinoma virus, Canine coronavirus (CCV), pantropic CCV, Canine respiratory coronavirus, Bovine coronavirus, Feline Calicivirus, Feline enteric coronavirus, Feline infectious peritonitis, virus, Porcine epidemic diarrhea virus, Porcine hemagglutinating encephalomyletitis virus, Porcine parvovirus, Porcine Circovirus (PCV) Type I, PCV Type II, Porcine Reproductive and Respiratory Syndrome (PRRS) Virus, Transmissible gastroenteritis virus, Turkey coronavirus, Bovine ephemeral fever virus, Rabies, Rotovirus, Vesicular stomatitis virus, lentivirus, Avian influenza, Rhinoviruses, Equine influenza virus, Swine influenza virus, Canine influenza virus, Feline influenza virus, Human influenza virus, Eastern Equine encephalitis virus (EEE), Venezuelan equine encephalitis virus, West Nile virus, Western equine encephalitis virus, human immunodeficiency virus, human papilloma virus, varicella zoster virus, hepatitis B virus, rhinovirus, and measles virus, and combinations thereof.

Examples of parasites causing disease for which immune responsiveness may be obtained include, for example, Anaplasma, Fasciola hepatica (liver fluke), Coccidia, Eimeria spp., Neospora caninum, Toxoplasma gondii, Giardia, Dirofilaria (heartworms), Ancylostoma (hookworms), Trypanosoma spp., Leishmania spp., Trichomonas spp., Cryptosporidium parvum, Babesia, Schistosoma, Taenia, Strongyloides, Ascaris, Trichinella, Sarcocystis, Hammondia, and Isopsora, and combinations thereof. Also contemplated are external parasites including, but not limited to, ticks, including Ixodes, Rhipicephalus, Dermacentor, Amblyomma, Boophilus, Hyalomma, and Haemaphysalis species, and combinations thereof.

The methods of the disclosure find use with various classes of vaccines. Some vaccines contain live, attenuated microorganisms. Many of these are active viruses that have been cultivated under conditions that disable their virulent properties, or that use closely related but less dangerous organisms to produce a broad immune response. Examples of attenuated vaccines include the yellow fever, polio, measles, mumps, and rubella (MMR), varicella zoster virus (chicken pox), rotavirus, variola (smallpox), and the bacterial diseases: typhoid, Mycobacterium tuberculosis, Yersinia pestis, etc.

Inactivated vaccines include, for example, IPV (polio vaccine), hepatitis A vaccine, rabies vaccine and most influenza vaccines.

Toxoid vaccines are made from inactivated toxic compounds that cause illness rather than the micro-organism. Examples of toxoid-based vaccines include tetanus and diphtheria, and Crotalus atrox toxoid is used to vaccinate dogs against rattlesnake bites.

Subunit vaccines uses a fragment of a pathogen to create an immune response, e.g. a polysaccharide, proteins, virus-like particles, etc. One example is the subunit vaccine against hepatitis B, which is composed of only the surface proteins of the virus. Another example is the virus-like particle (VLP) vaccine against human papillomavirus (HPV), which is composed of the viral major capsid protein. Another example is the hemagglutinin and neuraminidase subunits of the influenza virus. A subunit vaccine is being used for yersinia vaccination. Other subunit vaccines include Hib (Haemophilus influenzae type b) disease, whooping cough (part of the DTaP combined vaccine), pneumococcal disease (including pneumococcal polysaccharide vaccine (PPSV23) and pneumococcal conjugate vaccine (PCV13)), meningococcal disease, and shingles.

Genetic vaccines are based on the principle of uptake of a nucleic acid into cells, whereupon a protein is produced according to the nucleic acid template. This protein is usually the immunodominant antigen of the pathogen or a surface protein that enables the formation of neutralizing antibodies. The subgroup of genetic vaccines encompass viral vector vaccines, RNA vaccines and DNA vaccines.

Viral vector vaccines use a safe virus to insert pathogen genes in the body to produce specific antigens, such as surface proteins, to stimulate an immune response. Several different viruses have been used as vectors, including influenza, vesicular stomatitis virus (VSV), measles virus, and adenovirus. Adenovirus is one of the viral vectors used in some COVID-19 vaccines.

An mRNA vaccine (or RNA vaccine) is a novel type of vaccine which is composed of the nucleic acid RNA, packaged within a vector such as lipid nanoparticles. Among the COVID-19 vaccines are a number of RNA vaccines to combat the COVID-19 pandemic and some have been approved or have received emergency use authorization in some countries. For example, the Pfizer-BioNTech vaccine and Moderna mRNA vaccine are approved for use in adults and children in the US.

Immunogens in current use include, for example, acellular pertussis, Bacillus Calmette-Guérin, cholera, diphtheria, enterotoxigenic Escherichia coli, Ebola virus, Haemophilus influenzae type b, hepatitis A, hepatitis B, human papillomavirus, influenza, inactivated poliomyelitis, Japanese encephalitis, meningococcus, Neisseria meningitidis Serogroup B protein, measles, mumps, pneumococcus, rabies, rotavirus, respiratory syncytial virus, rubella, SARS-COV-2, tetanus, typhoid, Vibrio cholerae serogroup O1, varicella, yellow fever, etc.

The terms “biomarker,” “biomarkers,” “marker” or “markers” for the purposes of the invention refer to, without limitation, proteins together with their related metabolites, mutations, variants, polymorphisms, modifications, fragments, subunits, degradation products, elements, and other analytes or sample-derived measures. Markers include expression levels of a gene of interest. Markers can also include combinations of any one or more of the foregoing measurements, including temporal trends and differences. Broadly used, a marker can also refer to an immune cell subset.

To “analyze” includes determining a set of values associated with a sample by measurement of a marker (such as, e.g., presence or absence of a marker or constituent expression levels) in the sample and comparing the measurement against measurement in a sample or set of samples from the same subject or other control subject(s). The markers of the present teachings can be analyzed by any of various conventional methods known in the art. To “analyze” can include performing a statistical analysis, e.g. normalization of data, determination of statistical significance, determination of statistical correlations, clustering algorithms, and the like.

A “sample” in the context of the present teachings refers to any biological sample that is isolated from a subject, generally a sample comprising circulating immune cells. A sample can include, without limitation, an aliquot of body fluid, whole blood, PBMC (white blood cells or leucocytes), tissue biopsies, synovial fluid, lymphatic fluid, ascites fluid, and interstitial or extracellular fluid. “Blood sample” can refer to whole blood or a fraction thereof, including blood cells, white blood cells or leucocytes. Samples can be obtained from a subject by means including but not limited to venipuncture, biopsy, needle aspirate, lavage, scraping, surgical incision, or intervention or other means known in the art.

The term “antibody” includes full length antibodies and antibody fragments, and can refer to a natural antibody from any organism, an engineered antibody, or an antibody generated recombinantly for experimental, therapeutic, or other purposes as further defined below. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports, and possess other variations.

An antibody response of interest for the present disclosure relates to the production of specific antibodies in response to exposure to an immunogen, resulting in the presence of such antibodies in the subject blood, including serum. Of interest is the titer, or level, of such specific antibodies; and the duration of response. Antibodies may be of various classes, e.g. IgM, IgG, IgA, IgE, etc. In some embodiments a mature, IgG response is of interest.

The endpoints for immunogenicity may be a determination of antibody titers. The antibody titer may be determined at one or more suitable time points. For example, the peak antibody titer may be determine at around 6 to about 8 weeks post immunization, e.g. at around 35 to about 45 days, at around 42 days, etc. The residual antibody titer may be determined at a later time point, for example from about 4 to about 8 months post immunization, from about 5 to about 7 months post immunization, and may be around 6 months post immunization, e.g. around 150 to about 200 days post immunization.

The amount of specific antibody present in blood, polyclonal antiserum, etc. can be quantitated by various assays, using serially diluted antiserum. The amount of specific antibody in the antiserum is determined from a standard curve generated with a specific antibody of known concentration. The unknown antiserum and the standard antibody are assayed in parallel.

Assays to determine specific antibody titer may include, for example, Enzyme-Linked Immunosorbent Assay (ELISA). ELISA is a highly sensitive assay that detects and quantifies antibodies in a sample. In this assay, antigens are immobilized on a solid surface (such as a microtiter plate), and antibodies in the sample bind to these antigens. Detection is usually achieved using an enzyme-conjugated secondary antibody that produces a colorimetric or fluorescent signal. The intensity of the signal is proportional to the concentration of antibodies in the sample.

Neutralization assays measure the ability of antibodies to neutralize the activity of a pathogen or toxin. Typically, the sample containing antibodies is mixed with the pathogen or toxin, and the mixture is then added to cultured cells or animals. The reduction or prevention of the pathogen's or toxin's activity indicates the presence of neutralizing antibodies.

Hemagglutination Inhibition (HI) Assay is commonly used for influenza virus antibodies. It measures the ability of antibodies to inhibit the agglutination of red blood cells (hemagglutination) by the virus. The titer is determined by the highest dilution of the serum that inhibits hemagglutination.

Similar to neutralization assays, microneutralization assays measure the ability of antibodies to neutralize virus infectivity. However, they are performed in microplate formats, often using cell culture systems, and typically involve endpoint titration to determine the highest dilution of serum that neutralizes viral infectivity.

Plaque Reduction Neutralization Test (PRNT) measures the ability of antibodies to neutralize viruses by reducing the number of plaques formed in cell culture. The serum is serially diluted and mixed with a known quantity of virus before being added to cell monolayers. The reduction in plaque formation is indicative of the presence of neutralizing antibodies.

Although less commonly used for antibody titration, Western blotting can be employed to confirm the presence of specific antibodies. In this technique, proteins from a sample are separated by electrophoresis, transferred to a membrane, and then probed with antibodies. The presence of specific bands indicates the presence of antibodies against particular antigens.

These assays can be tailored to measure antibody titers against specific pathogens or antigens and are crucial for assessing the immune response following vaccination or infection. Choosing the appropriate assay depends on factors such as the nature of the pathogen, the type of antibodies being measured, and the desired level of sensitivity and specificity.

The present invention incorporates information disclosed in other applications and texts. The following patent and other publications are hereby incorporated by reference in their entireties: Alberts et al., The Molecular Biology of the Cell, 4th Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7th Ed., Garland, and Leroith and Bondy, Growth Factors and Cytokines in Health and Disease, A Multi Volume Treatise, Volumes 1A and IB, Growth Factors, 1996.

Unless otherwise apparent from the context, all elements, steps or features of the invention can be used in any combination with other elements, steps or features.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Due to biological functional equivalency considerations, changes can be made in protein structure without affecting the biological action in kind or amount. All such modifications are intended to be included within the scope of the appended claims.

The subject methods are used for prophylactic or therapeutic purposes. As used herein, the term “treating” is used to refer to both prevention of relapses, and treatment of pre-existing conditions. For example, the development of immunity can be accomplished by administration of the agent. The treatment of ongoing disease, where the treatment stabilizes or improves the clinical symptoms of the patient, is of particular interest.

### Compositions and Methods of the Invention

The term “immunostimulatory composition” refers to a composition that of an antigen suitably formulated for administration to an individual, and may be more conventionally referred to as a vaccine. Administration of the composition to a subject results in an immune response, which by be a B cell (antibody) or T cell response. The amount of a composition that is therapeutically effective may vary depending on the dose of antigen, the presence of adjuvant, and the condition of the subject, and can be determined by one skilled in the art.

As shown herein, co-administration of an effective dose of a thrombospondin agent, e.g. thrombopoietin protein; mRNA- or DNA-based vectors expressing thrombopoietin; small or large molecules that activate thrombopoietic activity via cMPL including thrombopoietin receptor agonists (TPO-RA) such as eltrombopag, avatrombopag, lusutrombopag, hetrombopag, romiplostim; and the like, enhances the durability of an antibody response to an immunogen, where the titer of specific antibodies to the antigen are present for an extended period of time. The increase in durability can be determined, for example, by measuring the residual, i.e. day 180 titer relative to day 42, or peak, titer. The residual may be a positive number with the inclusion of a thrombopoietin agent, e.g. at least 0.5, at least 1, at least 2, up to 4 or 4. The thrombopoietin agent may be co-formulated in a vaccine composition, or may be separately administered.

“Thrombopoietin” (TPO) is a glycoprotein hormone produced by the liver and kidney that regulates the production of platelets. It stimulates the production and differentiation of megakaryocytes. TPO is the ligand for the mpl receptor, CD110. The refseq for human thrombopoietin may be accessed at Genbank: NP_000451, NP_001171068, NP_001171069, NP_001276926, or NP_001276927.

Recombinant human thrombopoietin has shown clinical benefit but some immunogenicity, therefore second-generation agents were designed to be sufficiently structurally unique from endogenous TPO to avoid development of TPO-reactive antibodies and to have different pharmacologic attributes. The first of these was the peptibody romiplostim, which was created by combining the IgG1 Fc portion with four 14-amino acid peptides. This recombinant protein has a binding affinity comparable to that of endogenous TPO for the TPO-R and competes with endogenous TPO for TPO-R occupancy. Romiplostim was followed by small-molecule TPO-R agonists (TPO-RAs) eltrombopag, avatrombopag, and lusutrombopag, all of which bind to the same site in the transmembrane domain of the TPO-R, a region distinctly different from that where TPO and romiplostim bind. Currently, all of these TPO-RAs are widely used in multiple clinical areas. Hetrombopag is another similar oral TPO-RA.

The dose of a TPO-RA in the methods of the disclosure may be comparable to conventional dosing, or may be reduced or increased, e.g. up to the maximum approved dose. For example, the dose may be about 10%, about 25%, about 50%, about 75%, about 90%, about 125%, about 150%, about 200% of the conventional prescribed dose.

Conventional dosing with eltrombopag starts at 50 mg daily, unless the patient is East Asian in whom a lower dose should initially be used. If a response is not seen in two weeks, the dose is increased to 75 mg daily. Conventional dosing of romiplostim recommends 1 μg/kg/week and increasing by 1 μg/kg/week until a response is achieved, but may start at 3 μg/kg/week, particularly if a rapid response is needed, or one full vial of 250 μg, and increasing weekly to 5, 7, and then 10 μg/kg/week until a response is achieved. Median dose of romiplostim in adults is 3-5 μg/kg/week. For avatrombopag, conventional dosing is oral at 20 mg, initiated 10-13 days before a scheduled procedure. For lusutrombopag, dosing is a 3 mg tablet initiated 8-14 days prior to a scheduled procedure. Hetrombopag has been administered as a single dose in five dose cohorts (5 mg, 10 mg, 20 mg, 30 mg or 40 mg); or given once daily for 10 days in three dose cohorts (2.5 mg, 5.0 mg or 7.5 mg).

The thrombopoietin agonist may be administered prior to the administration of an immunogen, concurrently with an immunogen, or following administration of an immunogen. In some embodiments the TPO agonist is administered from about 1, about 2, about 3, about 5, to about 10 days prior, and/or about about 1, about 2, about 3, about 5, to about 10 days following administration of the immunogen, and may be administered on the same day as the immunogen. An effective dose may be, for example, equivalent to from about 1 to 1000 μg/kg TPO protein.

The TPO agonist may be administered as a single dose or multiple doses, as appropriate for the specific agent, e.g. 1, 2, 3, 4, 5, or more doses. The TPO agonist may be administered in combination with an initial, “priming” dose of immunogen; may be administered in combination with one or more a booster doses of immunogen; or may be administered with both.

In some embodiments an adjuvant composition is included in the vaccine. Exemplary adjuvants are oild in water emulsions, and may comprise squalene in the oil phase. For example, AS03 is an adjuvant system composed of a-tocopherol, squalene and polysorbate 80 in an oil-in-water emulsion. MF59 is another immunologic adjuvant that comprises a squalene emulsion. The dose of adjuvant administered may depend on the antigen with which it is used and the antigen dosage to be applied. It is also dependent on the intended species and the desired formulation. Usually the quantity is within the range conventionally used for adjuvants. For example, adjuvants typically comprises from about 1 μg to about 1000 μg, inclusive, of a 1-mL dose.

The adjuvant formulations can be homogenized or microfluidized. The formulations are subjected to a primary blending process, typically by passage one or more times through one or more homogenizers. Any commercially available homogenizer can be used for this purpose, e.g., Ross emulsifier (Hauppauge, N.Y.), Gaulin homogenizer (Everett, Mass.), or Microfluidics (Newton, Mass.). In one embodiment, the formulations are homogenized for three minutes at 10,000 rpm. Microfluidization can be achieved by use of a commercial mirofluidizer, such as model number 110Y available from Microfluidics, (Newton, Mass.); Gaulin Model 30CD (Gaulin, Inc., Everett, Mass.); and Rainnie Minilab Type 8.30H (Miro Atomizer Food and Dairy, Inc., Hudson, Wis.). These microfluidizers operate by forcing fluids through small apertures under high pressure, such that two fluid streams interact at high velocities in an interaction chamber to form compositions with droplets of a submicron size. In one embodiment, the formulations are microfluidized by being passed through a 200 micron limiting dimension chamber at 10,000+/−500 psi.

The routes of administration for an immunostimulatory composition include parenteral, oral, oronasal, intranasal, intratracheal, topical, etc. Any suitable device may be used to administer the compositions, including syringes, droppers, needleless injection devices, patches, and the like. The route and device selected for use will depend on the composition of the adjuvant, the antigen, and the subject, and such are well known to the skilled artisan.

The immunostimulatory compositions can further include one or more immunomodulatory agents such as, e.g., quaternary ammonium compounds (e.g., DDA), and interleukins, interferons, or other cytokines. These materials can be purchased commercially. The amount of an immunomodulator suitable for use in the adjuvant compositions depends upon the nature of the immunomodulator used and the subject. However, they are generally used in an amount of about 1 μg to about 5,000 μg per dose.

The immunostimulatory compositions can further include one or more polymers such as, for example, DEAE Dextran, polyethylene glycol, and polyacrylic acid and polymethacrylic acid (eg, CARBOPOL. RTM.). Such material can be purchased commercially. The amount of polymers suitable for use in the adjuvant compositions depends upon the nature of the polymers used. However, they are generally used in an amount of about 0.0001% volume to volume (v/v) to about 75% v/v. In other embodiments, they are used in an amount of about 0.001% v/v to about 50% v/v, of about 0.005% v/v to about 25% v/v, of about 0.01% v/v to about 10% v/v, of about 0.05% v/v to about 2% v/v, and of about 0.1% v/v to about 0.75% v/V. In another embodiment, they are used in an amount of about 0.02 v/v to about 0.4% v/V. DEAE-dextran can have a molecular size in the range of 50,000 Da to 5,000,000 Da, or it can be in the range of 500,000 Da to 2,000,000 Da. Such material may be purchased commercially or prepared from dextran.

The immunostimulatory compositions can further include one or more Th2 stimulants such as, for example, Bay R1005™ and aluminum. The amount of Th2 stimulants suitable for use in the adjuvant compositions depends upon the nature of the Th2 stimulant used. However, they are generally used in an amount of about 0.01 mg to about 10 mg per dose. In other embodiments, they are used in an amount of about 0.05 mg to about 7.5 mg per dose, of about 0.1 mg to about 5 mg per dose, of about 0.5 mg to about 2.5 mg per dose, and of 1 mg to about 2 mg per dose. A specific example is Bay R1005™, a glycolipid with the chemical name “N-(2-deoxy-2-L-leucylamino-β-D-glucopyranosyl)-N-octadecyldodecanamide acetate.” It is an amphiphilic molecule which forms micelles in aqueous solution.

Some examples of viruses that provide a source of antigens for a vaccine include, for example, Aceinetobacter calcoaceticus, Acetobacter paseruianus, Actinobacillus pleuropneumoniae, Aeromonas hydrophila, Alicyclobacillus acidocaldarius, Arhaeglobus fulgidus, Bacillus pumilus, Bacillus stearothermophillus, Bacillus subtilis, Bacillus thermocatenulatus, Bordetella bronchiseptica, Burkholderia cepacia, Burkholderia glumae, Campylobacter coli, Campylobacter fetus, Campylobacter jejuni, Campylobacter hyointestinalis, Chlamydia psittaci, Chlamydia trachomatis, Chlamydophila spp., Chromobacterium viscosum, Erysipelothrix rhusiopathieae, Listeria monocytogenes, Ehrlichia canis, Escherichia coli, Haemophilus influenzae, Haemophilus somnus, Helicobacter suis, Lawsonia intracellularis, Legionella pneumophilia, Moraxellsa sp., Mycobactrium bovis, Mycoplasma hyopneumoniae, Mycoplasma mycoides subsp. mycoides LC, Clostridium perfringens, Odoribacter denticanis, Pasteurella (Mannheimia ) haemolytica, Pasteurella multocida, Photorhabdus luminescens, Porphyromonas gulae, Porphyromonas gingivalis, Porphyromonas salivosa, Propionibacterium acnes, Proteus vulgaris, Pseudomnas wisconsinensis, Pseudomonas aeruginosa, Pseudomonas fluorescens C9, Pseudomonas fluorescensSIKW1 , Pseudomonas fragi, Pseudomonas luteola, Pseudomonas oleovorans, Pseudomonas sp B11-1, Alcaliges eutrophus, Psychrobacter immobilis, Rickettsia prowazekii, Rickettsia rickettsia, Salmonella typhimurium, Salmonella bongori, Salmonella enterica, Salmonella dublin, Salmonella typhimurium, Salmonella choleraseuis, Salmonella newport, Serratia marcescens, Spirlina platensis, Staphlyoccocus aureus, Staphylococcus epidermidis, Staphylococcus hyicus, Streptomyces albus, Streptomyces cinnamoneus, Streptococcus suis, Streptomyces exfoliates, Streptomyces scabies, Sulfolobus acidocaldarius, Syechocystis sp., Vibrio cholerae, Borrelia burgdorferi, Treponema denticola, Treponema minutum, Treponema phagedenis, Treponema refringens, Treponema vincentii, Treponema palladium, and Leptospira species, such as the known pathogens Leptospira canicola, Leptospira grippotyposa, Leptospira hardjo, Leptospira borgpetersenii hardjo-bovis, Leptospira borgpetersenii hardjo-prajitno, Leptospira interrogans, Leptospira icterohaemorrhagiae, Leptospira pomona, and Leptospira bratislava, and combinations thereof.

Examples of viruses that provide a source of antigens for a vaccine include, for example, SARS-Cov1, SARS-Cov2, and other coronaviruses, Avian herpesviruses, Bovine herpesviruses, Canine herpesviruses, Equine herpesviruses, Feline viral rhinotracheitis virus, Marek's disease virus, Ovine herpesviruses, Porcine herpesviruses, Pseudorabies virus, Avian paramyxoviruses, Bovine respiratory syncytial virus, Canine distemper virus, Canine parainfluenza virus, canine adenovirus, canine parvovirus, Bovine Parainfluenza virus 3, Ovine parainfluenza 3, Rinderpest virus, Border disease virus, Bovine viral diarrhea virus (BVDV), BVDV Type I, BVDV Type II, Classical swine fever virus, Avian Leukosis virus, Bovine immunodeficiency virus, Bovine leukemia virus, Bovine tuberculosis, Equine infectious anemia virus, Feline immunodeficiency virus, Feline leukemia virus (FeLV), Newcastle Disease virus, Ovine progressive pneumonia virus, Ovine pulmonary adenocarcinoma virus, Canine coronavirus (CCV), pantropic CCV, Canine respiratory coronavirus, Bovine coronavirus, Feline Calicivirus, Feline enteric coronavirus, Feline infectious peritonitis, virus, Porcine epidemic diarrhea virus, Porcine hemagglutinating encephalomyletitis virus, Porcine parvovirus, Porcine Circovirus (PCV) Type I, PCV Type II, Porcine Reproductive and Respiratory Syndrome (PRRS) Virus, Transmissible gastroenteritis virus, Turkey coronavirus, Bovine ephemeral fever virus, Rabies, Rotovirus, Vesicular stomatitis virus, lentivirus, Avian influenza, Rhinoviruses, Equine influenza virus, Swine influenza virus, Canine influenza virus, Feline influenza virus, Human influenza virus, Eastern Equine encephalitis virus (EEE), Venezuelan equine encephalitis virus, West Nile virus, Western equine encephalitis virus, human immunodeficiency virus, human papilloma virus, varicella zoster virus, hepatitis B virus, rhinovirus, and measles virus, and combinations thereof.

Examples of parasites that provide a source of antigens for a vaccine include, for example, Anaplasma, Fasciola hepatica (liver fluke), Coccidia, Eimeria spp., Neospora caninum, Toxoplasma gondii, Giardia, Dirofilaria (heartworms), Ancylostoma (hookworms), Trypanosoma spp., Leishmania spp., Trichomonas spp., Cryptosporidium parvum, Babesia, Schistosoma, Taenia, Strongyloides, Ascaris, Trichinella, Sarcocystis, Hammondia, and Isopsora, and combinations thereof. Also contemplated are external parasites including, but not limited to, ticks, including Ixodes, Rhipicephalus, Dermacentor, Amblyomma, Boophilus, Hyalomma, and Haemaphysalis species, and combinations thereof.

Oil, when added as a component, generally provides a long and slow release profile. In the present invention, the oil can be metabolizable or non-metabolizable. The oil can be in the form of an oil-in-water, a water-in-oil, or a water-in-oil-in-water emulsion. Oils suitable for use in the present invention include alkanes, alkenes, alkynes, and their corresponding acids and alcohols, the ethers and esters thereof, and mixtures thereof. The individual compounds of the oil are light hydrocarbon compounds, i.e., such components have 6 to 30 carbon atoms. The oil can be synthetically prepared or purified from petroleum products. The moiety may have a straight or branched chain structure. It may be fully saturated or have one or more double or triple bonds. Some non-metabolizable oils for use in the present invention include mineral oil, paraffin oil, and cycloparaffins, for example. The term oil is also intended to include “light mineral oil,” i.e., oil which is similarly obtained by distillation of petrolatum, but which has a slightly lower specific gravity than white mineral oil.

Metabolizable oils include metabolizable, non-toxic oils. The oil can be any vegetable oil, fish oil, animal oil or synthetically prepared oil which can be metabolized by the body of the subject to which the adjuvant will be administered and which is not toxic to the subject. Sources for vegetable oils include nuts, seeds and grains.

Other components of the compositions can include pharmaceutically acceptable excipients, such as carriers, solvents, and diluents, isotonic agents, buffering agents, stabilizers, preservatives, vaso-constrictive agents, antibacterial agents, antifungal agents, and the like. Typical carriers, solvents, and diluents include water, saline, dextrose, ethanol, glycerol, oil, and the like. Representative isotonic agents include sodium chloride, dextrose, mannitol, sorbitol, lactose, and the like. Useful stabilizers include gelatin, albumin, and the like.

Surfactants are used to assist in the stabilization of an emulsion selected to act as the carrier for the adjuvant and antigen. Surfactants suitable for use in the present inventions include natural biologically compatible surfactants and non-natural synthetic surfactants. Biologically compatible surfactants include phospholipid compounds or a mixture of phospholipids. Preferred phospholipids are phosphatidylcholines (lecithin), such as soy or egg lecithin. Lecithin can be obtained as a mixture of phosphatides and triglycerides by water-washing crude vegetable oils, and separating and drying the resulting hydrated gums. A refined product can be obtained by fractionating the mixture for acetone insoluble phospholipids and glycolipids remaining after removal of the triglycerides and vegetable oil by acetone washing. Alternatively, lecithin can be obtained from various commercial sources. Other suitable phospholipids include phosphatidylglycerol, phosphatidylinositol, phosphatidylserine, phosphatidic acid, cardiolipin, and phosphatidylethanolamine. The phospholipids may be isolated from natural sources or conventionally synthesized.

Non-natural, synthetic surfactants suitable for use in the present invention include sorbitan-based non-ionic surfactants, e.g. fatty-acid-substituted sorbitan surfactants, fatty acid esters of polyethoxylated sorbitol (TWEEN™), polyethylene glycol esters of fatty acids from sources such as castor oil; polyethoxylated fatty acid, polyethoxylated isooctylphenol/formaldehyde polymer, polyoxyethylene fatty alcohol ethers (BRIJ™); polyoxyethylene nonphenyl ethers (TRITON™), polyoxyethylene isooctylphenyl ethers (TRITON™).

As used herein, “a pharmaceutically-acceptable carrier” includes any and all solvents, dispersion media, coatings, adjuvants, stabilizing agents, diluents, preservatives, antibacterial and antifungal agents, isotonic agents, adsorption delaying agents, and the like. The carrier(s) must be “acceptable” in the sense of being compatible with the other components of the compositions and not deleterious to the subject. Typically, the carriers will be will be sterile and pyrogen-free, and selected based on the mode of administration to be used. It is well known by those skilled in the art that the preferred formulations for the pharmaceutically acceptable carrier which comprise the compositions are those pharmaceutical carriers approved in the applicable regulations promulgated by the United States (US) Department of Agriculture or US Food and Drug Administration, or equivalent government agency in a non-US country. Therefore, the pharmaceutically accepted carrier for commercial production of the compositions is a carrier that is already approved or will be approved by the appropriate government agency in the US or foreign country.

The compositions optionally can include compatible pharmaceutically acceptable (i.e., sterile or non-toxic) liquid, semisolid, or solid diluents that serve as pharmaceutical vehicles, excipients, or media. Diluents can include water, saline, dextrose, ethanol, glycerol, and the like. Isotonic agents can include sodium chloride, dextrose, mannitol, sorbitol, and lactose, among others. Stabilizers include albumin, among others.

The compositions can also contain antibiotics or preservatives, including, for example, gentamicin, merthiolate, or chlorocresol. The various classes of antibiotics or preservatives from which to select are well known to the skilled artisan.

Kits may be provided. Kits may further include cells or reagents suitable for isolating and culturing cells in preparation for conversion; reagents suitable for culturing T cells; and reagents useful for determining the epigenomic effect of a vaccine adjuvant. Kits may also include tubes, buffers, etc., and instructions for use.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLE 1

The limited duration of humoral responses to vaccination is a key issue in the fight against infectious diseases, as antibody levels wane over time, leaving individuals vulnerable to reinfection. This disclosure addresses this problem by providing a method: (1) to assess the durability of waning antibody responses and (2) to enhance the durability of antibody responses to vaccination, by increasing thrombopoietic activity through the administration of thrombopoietin protein, mRNA-based, or DNA-based vectors expressing thrombopoietin, small or large molecules that activate thrombopoietic activity via cMPL. By assessing and enhancing both the magnitude and durability of antibody responses to vaccination, this disclosure provides for long-term protection against infectious diseases, addressing a critical need in the field.

Thrombopoietic activity refers to the physiological process by which the body produces platelets. During thrombopoiesis, immature megakaryocytes increase their ploidy and differentiate into mature polyploid megakaryocytes, which then undergo cytoplasmic fragmentation to give rise to small anucleate cell fragments called platelets. Newly generated platelets preserve RNA content from megakaryocytes, thus are named reticulated platelets. As platelets mature, cytosolic RNA gradually degrades, resulting in mature RNA-free platelets.

Thrombopoietin (TPO) is the primary cytokine that regulates thrombopoietic activity. TPO binds to its receptor, c-Mpl, on the surface of megakaryocytes, leading to activation of signaling pathways that promote the proliferation, differentiation, and cytoplasmic fragmentation of megakaryocytes.

We demonstrate: (1) a simple flow cytometry-based assay using blood samples to define a cell-based signature of durability and (2) that increasing thrombopoietic activity enhances the durability of antibody response to vaccination.

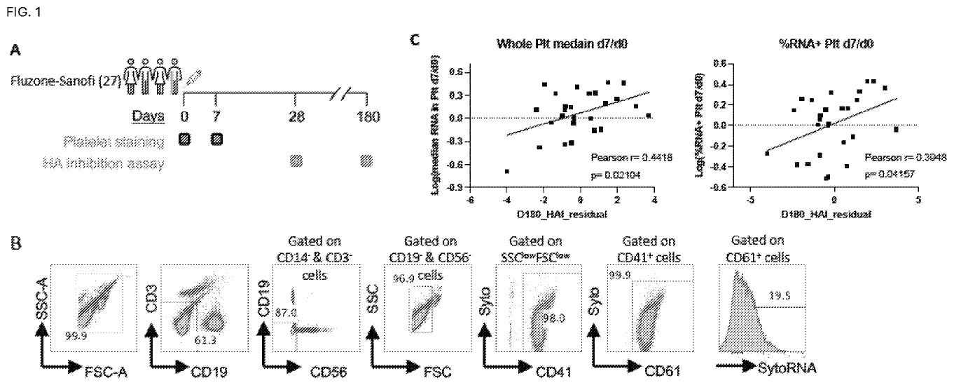

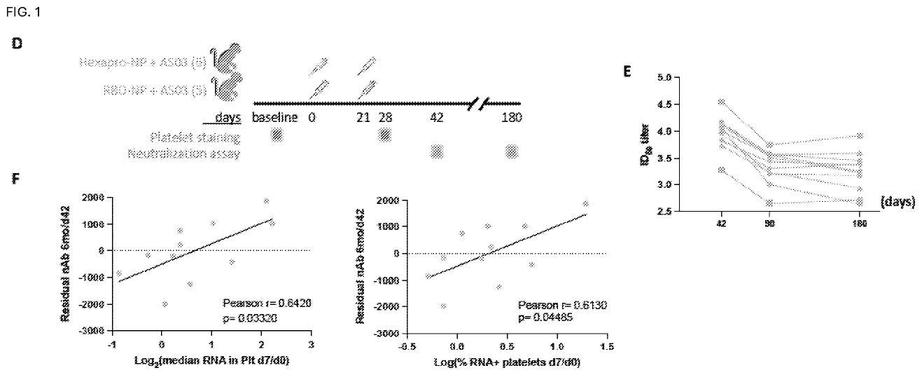

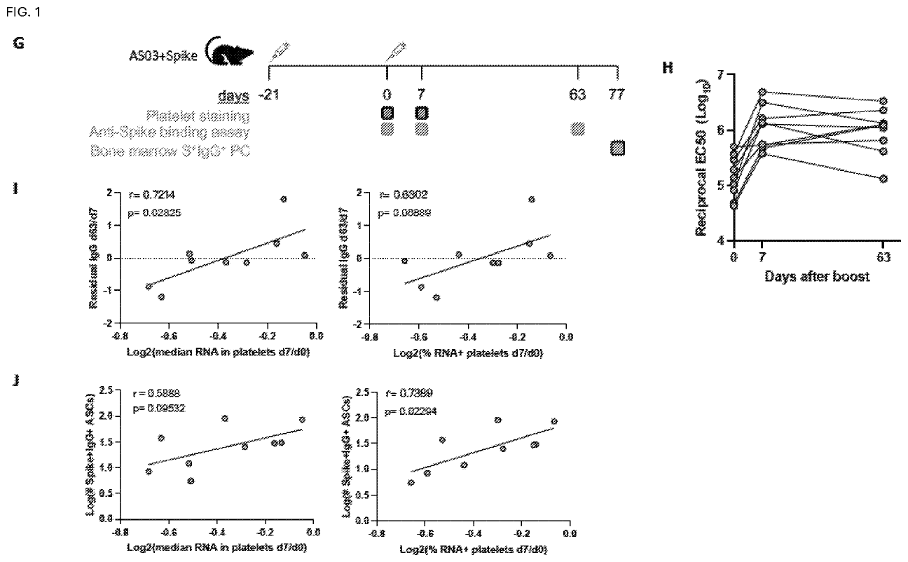

Flow cytometric analysis was used to assess the RNA content in platelets from subjects immunized with TIV during the 2010-11 influenza season (FIG. 1A). FSC/SSC dot plots of thawed PBMC samples showed CD41⁺CD61⁺ platelet populations, which are characterized by low SSC and FSC values and can be easily distinguished from PBMCs by size (FIG. 1B). Platelet frequencies in these samples were comparable to whole PBMCs and did not differ between days 0 and 7 post TIV vaccination. Importantly, the fold change of RNA content in whole platelets and % RNA⁺ platelets at day 7 versus day 0 in vaccinees positively correlated to the persistence of the antibody response, as measured by the “residual” day 180/day 42 (FIG. 1C). The residual is the difference between observed antibody titers at day 180 and the antibody titers predicted at day 180 based on the simple linear correlation between the day 42 titers (peak) and day 180 titers. This confirms the association between platelet RNA content and persistence of antibody responses to vaccination and provide a simple FACS-based assay to predict the persistence of antibody response to vaccination.

To explore the mechanisms underlying this association in more detail, we next examined responses to COVID-19 subunit RBD-NP or Hexa-Pro-NP AS03 immunization in Rhesus macaques (FIG. 1D). Neutralizing antibody responses peaked at day 42 and then gradually declined (FIG. 1E). The fold changes of RNA content in whole platelets and % RNA+platelets at day 7 versus baseline were correlated with the day 180/day 42 residual (FIG. 1F).

As long-term antibody titers are predominately generated by long-lived plasma cells (LLPCs) that reside in the bone marrow, the frequency of bone marrow antigen-specific plasma cells can be an indicator of durable neutralizing antibody response. To support our findings in humans and NHPs, we used mice vaccinated with COVID-19 subunit Spike AS03 immunization to evaluate platelet RNA content (FIGS. 1G, 1H) and found that the fold change of platelet RNA content at day 7 was correlated with the day 63/day 7 residual (FIG. 11) or the number of bone marrow LLPCs at day 63 post boost (FIG. 1J).

Our results demonstrate that peripheral platelet RNA content reflects the status of megakaryocytes and thrombopoietic activity in the bone marrow contributing to LLPC survival, and identify platelet RNA content as a biomarker for predicting the durability of antibody responses to vaccines.

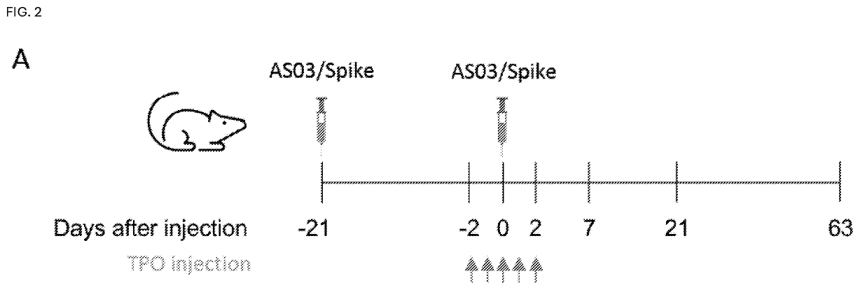

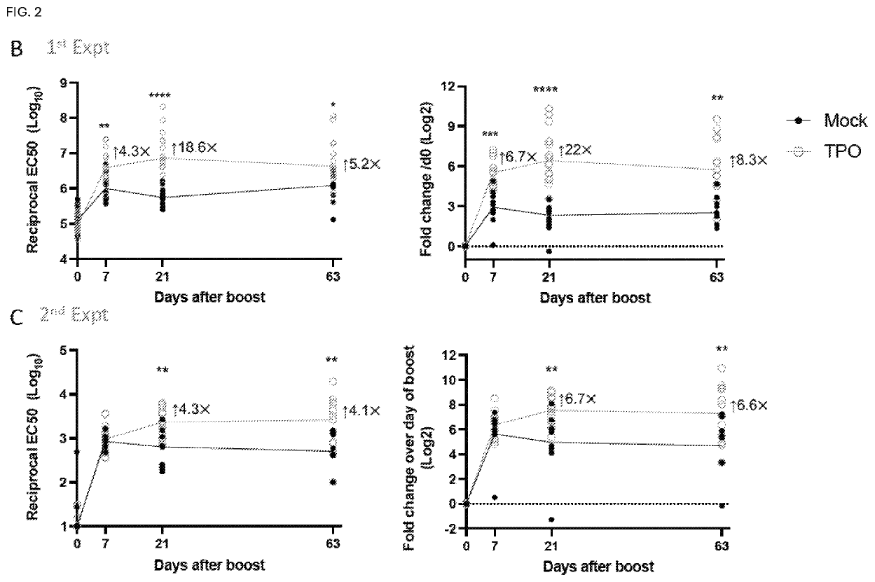

In order to investigate the direct impact of thrombopoietic activity on antibody persistence, we use recombinant mouse TPO to intraperitoneally inject mice within 5 days before and after the second immunization (FIG. 2A) to enhance the thrombopoietic activity 23 at the initial stage of boost. TPO administration significantly increased anti-Spike IgG tier at day 21 post boost and continued until day 63 (84-90 days after the first injection). Two independent experiments were shown in FIGS. 2B and 2C separately. Our data collectively show that enhancing thrombopoietic activity is a strategy for augmenting the magnitude and prolonging the duration of antibody responses to vaccines.

### Methods

FACS-based analysis of RNA content in platelets from human and non-human primate. PBMCs from human subjects in the 2010-2011 seasonal influenza vaccine (Fluzone-Sanofi) cohort were collected on the day of and day 7 post vaccination (Nakaya et al., Immunity 2015) and used for experiments. PBMCs from Rhesus macaques (Macaca mulatta) of Indian origin immunized twice with RBD-NP or HexaPro-NP AS03 vaccines, 21 days apart, were collected on the day of and day 7 after the second immunization (Arunachalam et al., Nature 2021) and used for experiments.

Previously cryopreserved PBMCs were thawed and clock-wisely gently dropped into 10 ml of complete RPMI medium. After centrifugation at 350×g for 5 min in room temperature, pellet was washed and resuspended in Phosphate buffer saline (PBS). One million cells were stained in 100 μl of PBS containing 1.5 μM SYTO™ RNASelect™ Green Fluorescent cell Stain (S32703, Invitrogen) as well as human-specific or non-human primate-specific antibody cocktails in room temperature. Twenty min later, 300 μl of 1% paraformaldehyde was directly added to samples. Cells were analyzed on a FACS Symphony flow cytometer (BD Biosciences) on the same day. The threshold for the FSC value was set to 4000 to ensure visualization of platelet population.

Analysis of flow cytometry files was performed using the FlowJo software (FlowJo, LLC). Human-specific antibody cocktail: anti-CD3-BUV737, anti-CD19-APC, anti-CD14-BV605, anti-CD56-PE, and anti-CD41-BV421, anti-CD61-PE-Cy7 antibodies. Platelets were defined as CD41⁺CD61⁺ cells after the exclusion of CD3⁺, CD19⁺, CD14⁺ and CD56⁺ cells. Non-human primate-specific antibody cocktail: anti-CD3-PE-CF594, anti-CD8-BUV563, anti-CD20-BUV737, anti-CD14-BUV805, and anti-CD41-BV421, anti-CD61-PE-Cy7 antibodies. Platelets were defined as CD41⁺ CD61⁺ cells after the exclusion of CD3⁺, CD8⁺, CD20⁺ and CD14⁺ cells.

Mice. C57BL/6 mice were purchased from Jackson Laboratories and bred in our animal facility at Stanford University. All mice in this study were matched for sex and aged between 8 and 14 weeks, maintained under specific pathogen-free conditions, a 12-h light/12-h dark cycle and temperatures of ~18-23 °C with 40-60% humidity and were handled according to the protocol approved by the Institutional Animal Care and Use Committee of Stanford University.

Spike AS03 formulations and immunizations. The SARS-COV-2 Spike Protein were produced from SinoBiological (Cat: 40589-V08B1). AS03 were kindly provided by GSK Vaccines, which is an oil-in-water emulsion containing 11.86 mg α-tocopherol, 10.69 mg sqyualene, and 4.86 mg polysorbate 80 (Tween-80) in PBS. For immunization, 1.5 μg of Spike protein was diluted in PBS to a final volume of 25 μl and mix with an equal volume of AS03. Each mouse was immunized in the right hind thigh muscle twice at an interval of 25 days using an Ultra-Fine™ Insulin Syringe (BD 320440) in a final 50 μL volume.

FACS-based analysis of RNA content in platelets from mouse. Mouse blood were collected on the day of or day 7 post the second immunization and anticoagulated with citrate-dextrose solution (sc-214744, Santa Cruz Biotechnology, Inc.) at a ratio of 6-8:1 was centrifugated at 150×g for 10 min in room temperature to obtain platelet-rich plasma. Thirteen ul of freshly prepared platelet-rich plasma was mixed with 40 μl of PBS containing 0.3 μl of anti-TER119-PE, anti-CD41-BV421, and anti-CD61-PE-Cy7 antibodies as well as 1.5 μM RNASelect™ Stain for 20 min at room temperature. At the end of staining, 200 μl of 1% paraformaldehyde was directly added to the sample. Mouse platelets were defined as CD41⁺ CD61⁺ cells after the exclusion of TER119⁺ red blood cells.

Thrombopoietic activity increasement. Lyophilized recombinant mouse thrombopoietin (TPO) was purchased from Peprotech (AF-315-14) or Gibco (RP-87753) and reconstituted at a final concentration of 0.5 mg/ml in endotoxin-free water. Two days before the second vaccine injection, each mouse was given 10 μg/kg of TPO diluted in 100 μL of PBS for 5 days.

Anti-Spike binding IgG ELISA. High-binding 96-well plates were coated with spike protein (2 μg/ml) diluted in PBS overnight and then washed by PBST (0.05% tween-20) three times. Wells were blocked with 3% non-fat milk in room temperature for 1 h. Serum samples were diluted 300-fold in the first row and serially diluted 5-fold in the following rows. Diluted samples were added to the plates and incubated at 37 °C for 1 h. After thoroughly wash with PBS-T, horseradish peroxidase-conjugated goat anti-mouse IgG (1:6,000, SouthernBiotech 1030-05) in 1% non-fat milk was added and incubated at room temperature for 1 h. ELISA plates were developed with 70 μL of 1-Step™ Ultra TMB-ELISA Substrate Solution (Thermo Scientific) and reactions were stopped with addition of 70 μL of 2N H₂SO₄. The optical density was measured at 450 nm with correction at 595 nm by subtraction with a Bio-Rad microplate reader.

This disclosure provides methods to specifically enhance the durability of antibody responses to vaccination. Adjuvants enhance the magnitude of the response but the effect on enhancing the durability is less clear. For example, in current studies, we used AS03 a potent vaccine adjuvant, yet the durability of the response induced by this could clearly be enhanced by TPO.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method for enhancing the durability of immune response to an immunostimulatory composition, the method comprising:
administering the immunostimulatory composition to a mammal in combination with administration of an effective dose of a thrombopoietin agent;
wherein the durability of at least an antibody response to the immunostimulatory composition is enhanced.

2. The method of claim 1, wherein the thrombopoietin agent is thrombopoietin protein, or a nucleic acid encoding thrombopoietin.

3. The method of claim 1, wherein the thrombopoietin agent is a thrombopoietin receptor agonist (TPO-RA).

4. The method of claim 3, wherein (TPO-RA) is selected from eltrombopag, avatrombopag, lusutrombopag, hetrombopag, and romiplostim.

5. The method of claim 1, wherein the thrombopoetin agent is administered with an initial priming dose of the immunostimulatory composition.

6. The method of claim 1, wherein the thrombopoetin agent is administered with a booster dose of the immunostimulatory composition.

7. The method of claim 1, wherein multiple doses of the thrombopoietin agent are administered.

8. The method of claim 1, wherein the thrombopoetin agent is co-formulated with the immunostimulatory composition.

9. The method of claim 1, wherein the thrombopoetin agent is separately formulated from the immunostimulatory composition.

10. The method of claim 9, wherein the thrombopoetin agent is administered with, or from about 1, about 2, about 3, about 5, to about 10 days prior, and/or about about 1, about 2, about 3, about 5, to about 10 days following administration of the immunostimulatory composition.

11. The method of claim 1, further comprising determining the specific antibody titer for the immunostimulatory composition at one or more time points.

12. The method of claim 11, wherein the durability residual is determined as the difference between observed antibody titers at day 180 and the antibody titers predicted at day 180 based on the simple linear correlation between the peak titers and day 180.

13. The method of claim 1, wherein the difference between observed antibody titers at day 180 and the antibody titers predicted at day 180 based on the simple linear correlation between the peak titers and day 180 titers, is a positive number.

14. The method of claim 1, wherein the mammal is a mouse, non-human primate, or human.

15. The method of claim 1, wherein the immunostimulatory composition comprises an adjuvant.

16. The method of any claim 1, wherein the immunostimulatory composition is an mRNA vaccine.

17. The method of claim 1, wherein the immunostimulatory composition is a viral vector vaccine.

18. The method of claim 1, wherein the immunostimulatory composition is a live or inactivated virus vaccine.

19. An immunostimulatory composition for use in the methods of claim 1.

20. A kit for use in the method of claim 1.
